(19) United States
(12) Patent Application Publication
(10) Pub. No.: US 2014/0039249 A1
(43) Pub. Date: Feb. 6, 2014

(54) VERY LIGHTWEIGHT SURGICAL MESH FOR VAGINAL PROLAPSE REPAIR

(71) Applicant: Rami Atalla, Welwyn (GB)

(72) Inventor: Rami Atalla, Welwyn (GB)

(21) Appl. No.: 13/983,401

(22) PCT Filed: Feb. 8, 2012
(86) PCT No.: PCT/GB12/00129

§ 371 Date: Oct. 17, 2013

(30) Foreign Application Priority Data
Feb. 8, 2011 (GB) 1102165.6
Mar. 1, 2011 (GB) 1103484.0

(51) Int. Cl. A61F 2/00

(52) U.S. Cl. CPC A61F 2/0063
USPC 600/37

(57) ABSTRACT

A surgical mesh for use in a surgical procedure for vaginal prolapse repair, the mesh including a first fiber or yarn and a second fiber or yarn, wherein the surgical mesh is knitted so that the first fiber or yarn is free to slide over the second fiber or yarn.

### FIELD OF THE INVENTION

The present invention relates to the field of surgical devices for vaginal prolapse repair, and in particular, to surgical meshes for use in surgical procedures for such vaginal and hernial repairs.

### BACKGROUND OF THE INVENTION

Prolapse of an internal organ through the vagina (e.g., cystocele, urethrocele, rectocele, enterocele, prolapsed uterus, vaginal vault prolapse) usually presents as a bulge in the vaginal walls. This often requires a surgical intervention in order to repair the vaginal prolapse, restore the prolapsed organ to its original position and maintain the vaginal walls in its anatomical position.

Surgeons use different techniques for repairing vaginal prolapse. The most common technique is the colporrhaphy. For example, for a cystocele, a vaginal wall is cut and separated from the bladder. The bladder is then pushed back in place and the pelvic floor tissues and fascia which have given way are stitched back to support the bladder.

Although this approach is widely used, the rate of recurrence when performing such operations is quite high. One of the reasons is the weakness of the pelvic floor and fascia, already damaged by the prolapsed organ. An alternative technique is the “site specific repair” used mainly in paravaginal defect repair, which involves re-approximating detached lateral vaginal tissue. Another technique involves the use of a prosthetic material to aid in support of the anterior or posterior vaginal walls, for example by using meshes or tension-free meshes.

A large variety of surgical meshes are now used by surgeons. They are usually classified by weight (heavyweight or lightweight) and by the size of the pores, i.e. the area between the fibres which form the meshes. In particular, small pore meshes have a pore diameter in the range of hundreds of microns (e.g., Marlex® produced by Bard Inc.) whereas large pore meshes have a pore diameter in the range of a few millimetres (e.g., Vypro® produced by Ethicon GmbH). Lightweight meshes usually have lower tensile strength than heavyweight meshes, whereas small pore meshes have less elasticity than large pore meshes. In general, lightweight meshes have less erosion and complications.

Surgical meshes for pelvic floor repair cause, however, a certain number of problems, such as foreign body reaction (FBR), vaginal mesh extrusion or exposure, pain (especially in the vagina during post-operation intercourse, the sensation of the presence of a mesh for both partners), infection, voiding dysfunction, recurrent prolapse and incontinence, shrinkage, chronic pain, infections, fistula and adhesion formation, calcification, degradation and erosion. Sometimes, such problems require complete removal of the surgical mesh originally inserted. This creates complications and discomfort in the affected patient. Such problems have even led certain health agencies to issue warnings about the use of the meshes currently on the market.

Accordingly, there is a need for a medical device which could assist in repairing a vaginal prolapse and minimize the problems caused by the currently available meshes.

### STATEMENT OF THE INVENTION

According to one aspect of the present invention, there is provided a very lightweight surgical mesh for use in a surgical procedure for vaginal prolapse repair or repair of hernia, the mesh including a first fibre or yarn and a second fibre or yarn, wherein the surgical mesh is so that the first fibre or yarn is free to slide over the second fibre or yarn. Preferably, the surgical mesh may have a porosity at rest of about 70% or more and, more preferably, the surgical mesh may have a porosity at rest of about 90% or more. The average diameter of a pore at rest may be about 1 centimetre or more, and, more preferably, the average diameter of a pore at rest may be about 1.1 centimetre or more. Also, the surgical mesh may be knitted so that the second fibre or yarn is able to slide over the first fibre or yarn.

According to a second aspect of the invention, there is provided a process for knitting a surgical mesh for use in a surgical procedure for vaginal prolapse repair or repair of hernia, the process including interweaving a first fibre or yarn with a second fibre or yarn so that the first fibre or yarn is free to slide over the second fibre or yarn.

According to a third aspect of the invention, there is provided a surgical mesh for use in a surgical procedure for vaginal prolapse repair or repair of hernia, the mesh including a first fibre or yarn and a second fibre or yarn, wherein the surgical mesh is knitted so that the first fibre or yarn is joined to the second fibre or yarn by a fixing arrangement or knot. The fixing arrangement or knot constrains a relative movement between the first fibre or yarn and the second fibre or yarn.

According to a fourth aspect of the invention, there is provided a process for knitting a surgical mesh for use in a surgical procedure for vaginal prolapse repair or repair of hernia, the process including interweaving a first fibre or yarn with a second fibre or yarn so that the first fibre or yarn is joined to the second fibre or yarn by a fixing arrangement.

According to a fifth aspect of the invention, there is provided a surgical mesh for use in a surgical procedure for vaginal prolapse repair or repair of hernia, the surgical mesh having a porosity at rest of 70% or more.

According to a sixth aspect of the invention, there is provided a surgical mesh for use in a surgical procedure for vaginal prolapse repair or repair of hernia, wherein the surgical mesh has a knitted border to maintain the fibres or yarn in place by distributing the pressures on the fibres of the mesh.

### BRIEF DESCRIPTION OF THE DRAWINGS

There now follows, by way of example only, a detailed description of embodiments of the present invention, with reference to the figures identified below.

FIG. 1 shows an example of a cystocele as described in the background of the invention;

FIG. 2 shows a surgical mesh in accordance with an embodiment of the present invention;

FIG. 3 shows a close-up view of a surgical mesh in accordance with another embodiment of the present invention;

FIGS. 4 A and B show a surgical mesh formed from a monofilament in accordance with an embodiment of the present invention; and

FIG. 5 shows some examples of possible fixing arrangements in accordance with an embodiment of the present invention.

### DETAILED DESCRIPTION OF THE EMBODIMENTS

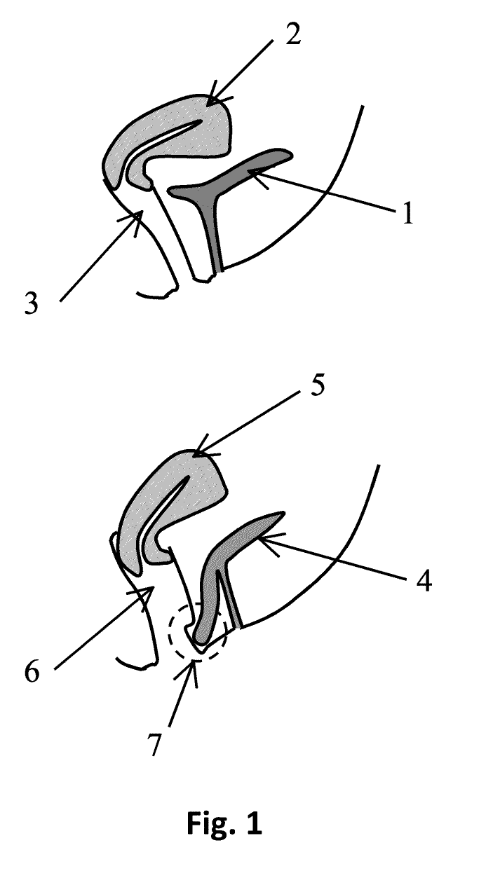

FIG. 1 shows an example of cystocele as discussed in the background of the invention. The top of FIG. 1 shows a normal situation, wherein the bladder (1) is in an anterior position with respect to the vagina (3), and the uterus (2) is in its normal position. The bottom of FIG. 1, instead, shows a situation in which a cystocele has occurred. The bladder (4) has prolapsed into the vagina (6) creating a bulge (7). In addition, the uterus (5) has moved downwards into the vagina (6). FIG. 1 only shows one of the possible conditions for which the medical device described in the present application is intended to be used. Other conditions include, for example, the conditions mentioned in the background to the invention in repair of vaginal, uterine prolapse or repair of hernia.

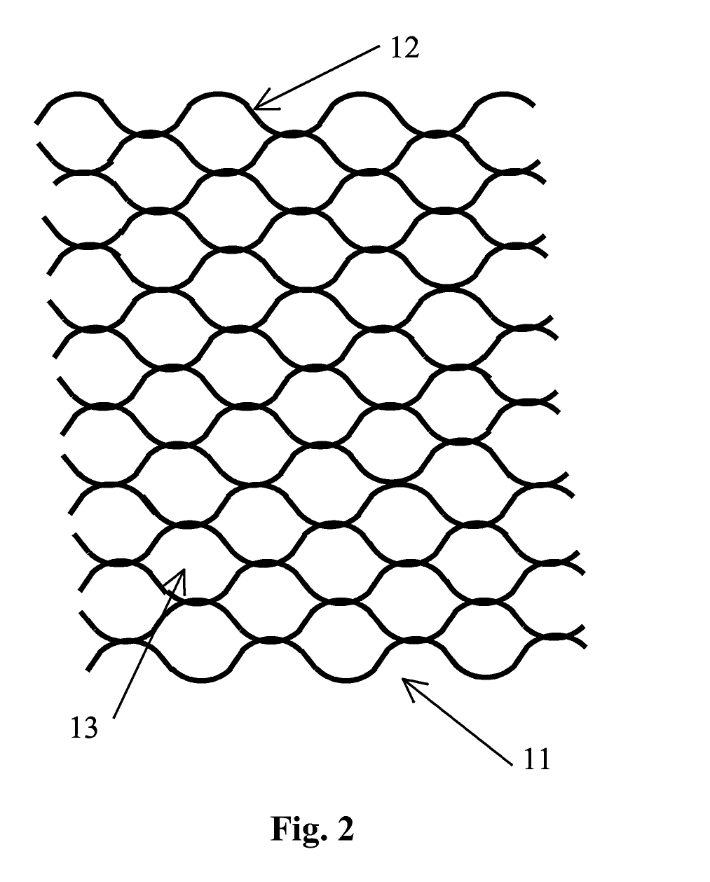

FIG. 2 shows a medical device in accordance with an embodiment of the present invention. The medical device is a surgical mesh (11) with a porosity, at rest, of about 70% or more. Preferably, the porosity is strictly greater than 75%. “Porosity” is defined as the area in the plane of the mesh not blocked by the fibres of the mesh. “At rest” is intended when the medical device is not implanted in the body and/or no forces (e.g., pulling or pushing forces, tearing forces, stresses, gravity) are applied to it.

The mesh is formed of fibres or yarns. The fibres (12) can be monofilament or multifilament. A yarn can be produced by interlocking two or more of said fibres. Each fibre is made of polypropylene (PP) or any other non-absorbable or absorbable material suitable for its use.

At rest, the average diameter of a pore (13) is about 1 centimetre or more. At rest, the pores can have a diamond shape, a square shape, a circular shape, any polygonal shape or any irregular shape acquired by the process of interweaving (or knitting) the fibres or yarns.

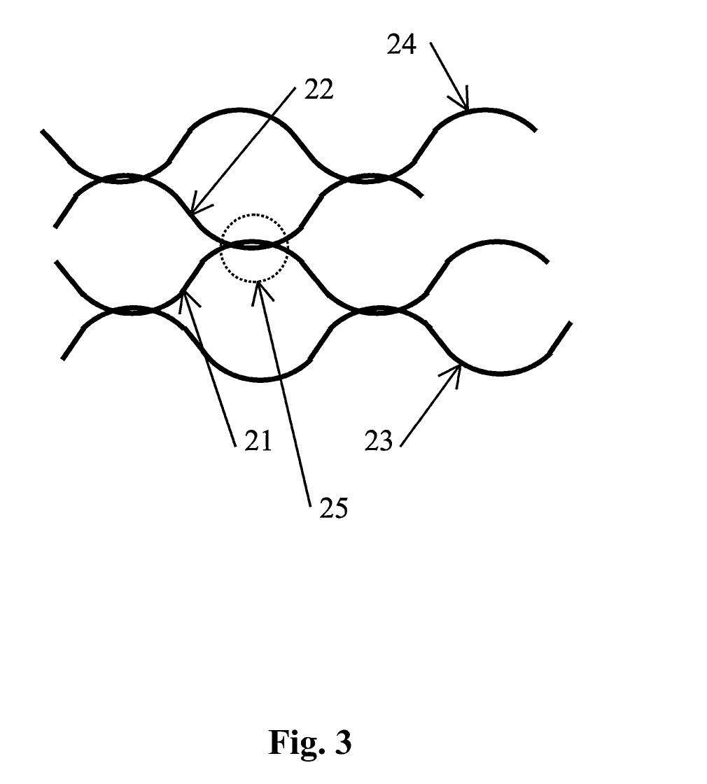

The mesh can be made by a knitting process. In particular, a first knitting process includes interweaving two or more fibres or yarns so that they are able to slide one over the other. An example of a resulting mesh is shown in FIG. 3, wherein fibre or yarn (21) is able to move across or over fibre or yarn (22), and/or fibre or yarn (22) is able to move across or over fibre or yarn (21), for example around the intersection area (25). A similar mechanism occurs between fibres or yarns (21) and (23), or between fibres or yarns (22) and (24). The direction of the weaving can vary between one row and another.

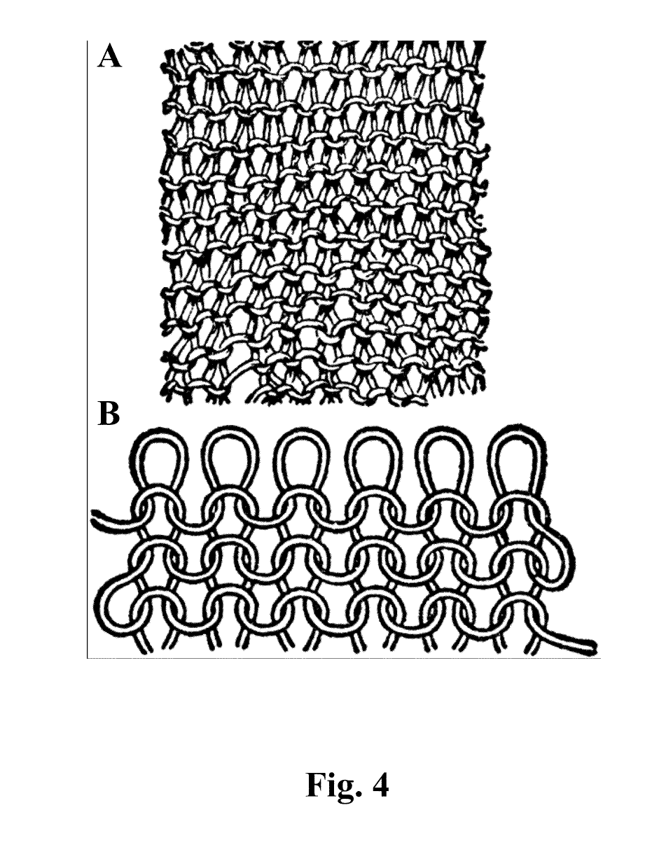

FIGS. 4 A and B show the another knitted pattern of the described surgical mesh formed from a monofilament. As can be seen from the mesh in FIG. 4A, the surgical mesh is composed of a single fibre or yarn which is knitted to form pores of the features as described above. A closer view of the pattern of the knitted monofilament can be seen in FIG. 4B

In addition, fibre or yarn (21) and fibre or yarn (22) may be joined by a fixing arrangement around the intersection area (25). A fixing arrangement may limit the ability of fibre or yarn (21) and fibre or yarn (22) to slide over one another.

In an alternative embodiment, a second knitting process includes interweaving two or more fibres or yarns so that the first fibre or yarn is joined to the second fibre or yarn by a fixing arrangement or knot. The fixing arrangement may constrain a relative movement between a first fibre or yarn and a second fibre or yarn. The fixing arrangement may limit or block a relative movement between a first fibre or yarn and a second fibre or yarn.

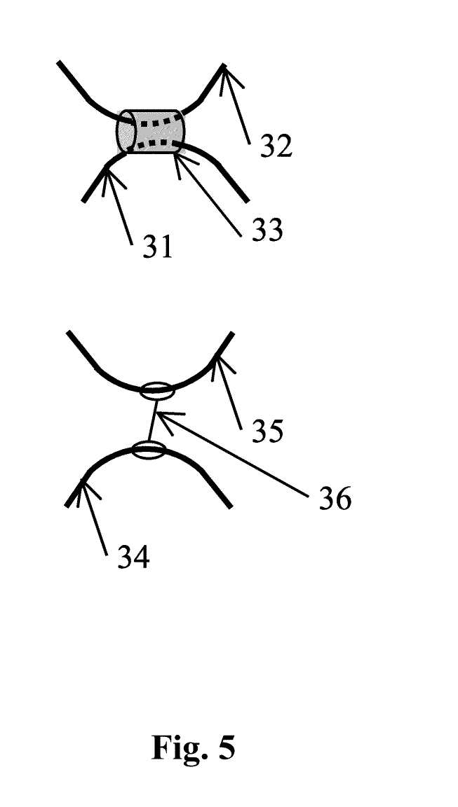

The fixing arrangement can be a simple knot of fibre or yarn (21) and fibre or yarn (22), a fastening element or a securing element. FIG. 5 shows some non-limiting examples of possible fixing arrangements. The fixing arrangement may be an element (33) that surrounds fibres or yarns (31) and (32) at the intersection area. This may allow fibres or yarns (31) and (32) (or, equivalently fibres or yarns (21) and (22) of FIG. 3) to be free to move with respect to one another without crossing over at the intersection area. It may also be used to secure, limit or block the movement of fibres or yarns (31) and (32) when they are crossed over at the intersection area. The fixing arrangement may also be a bridging element (36) that joins fibre or yarn (34) and fibre or yarn (35).

The device described above presents many advantages over the surgical meshes currently in use.

The mesh resulting from the first knitting process described above presents the advantage that the movement of each fibre or yarn with respect to one another adds flexibility to the mesh, and consequently advantageously improves the elasticity of the mesh (some of the advantages of an increased elasticity are discussed below).

In addition, each fibre or yarn is allowed to extend along its axis, thus increasing the stretchability of the mesh, which in turn is able to contain in a more efficient way any weight or pressure applied to it, such as a prolapsed organ, a vaginal wall or any object pressing on it. The mesh structure can contribute in guaranteeing that the fibres are not overstretched so as to reach their yield point. In this way, the overall chance of rupture or erosion of the mesh is also reduced.

The same can also be true for a mesh resulting from the second knitting process when the knot arrangement constrains without blocking a relative movement between a first fibre or yarn and a second fibre or yarn.

FBR depends on the surface area in contact with the host tissue, and the surface area strongly depends on textile properties such as the pore size or diameter and number of fibres/yarns used. A mesh in accordance with the invention reduces the surface area by a significant factor. Porosity, at rest, of about 70% or more (preferably strictly greater than 75%) and an average diameter of a pore (3), at rest, of about 1 centimetre or more imply that the surface area in contact with the host tissue is minimised. Moreover, larger pores will help in the healing process of the tissues as they allow more body tissue contact and hence faster healing.

This also benefits the healing process, as tissues can grow in a more efficient way. It thus reduces or eliminates the need of material and/or substance to help the healing processes, which is often required in meshes with a smaller porosity. Moreover, fibrotic reactions resulting from inflammatory response around the mesh is greatly reduced by the presence of minimal body (i.e., the material of the fibre).

Moreover, the decrease in weight of the mesh in accordance with the present invention, and hence the amount of foreign body involved in the repair, reduces the chance of rejection or exposure of the mesh.

The stiffness of the mesh is also greatly reduced. Tissue growth around the mesh causes the structure to lose elasticity because of the fibrotic bridgings created between the fibres/yarns. As a consequence, the mesh becomes more rigid, and is more likely to cause discomfort for one or both partners during intercourse. The surgical mesh provided by this invention considerably reduces the risk of losing elasticity, because the fibres are spaced apart from each other, thus reducing the occurrence of fibrotic bridging.

In addition, the structure of the mesh guarantees increased elasticity and reduces problems which are usually experienced during post-operation sexual intercourse. When incorporated in the pelvic floor, a mesh structure acts as a reinforcement for the pelvic floor, vaginal walls, or abdominal wall in case of hernia. This also has a consequence on the vaginal wall, which is able to sustain a greater pressure. However, when the mesh loses elasticity, the vaginal wall becomes less flexible. This can cause problems during intercourse when pressure is applied on the vaginal wall. Besides, as the thickness of the wall is relatively low, the stiff mesh can be felt, and there is a greater risk of vaginal exposure. The mesh structure in accordance with the invention greatly reduces these problems, since the elevated porosity guarantees greater elasticity and reduced stiffness, as described above. Thus, the reinforced vaginal wall will be more flexible to pressure, and at the same time the risk of exposing the mesh will be greatly reduced.

Moreover, all the above advantages are further achieved by the combination of the large porosity of the mesh and the freedom of movement of the fibres or yarns in the mesh guaranteed by the knitting process described above.

The overall shape at rest of the mesh will vary depending on the site of insertion. For instance, the shape of the mesh for the anterior vaginal wall will differ from the shape of the mesh for the posterior vaginal wall or from the shape of the mesh for the vault prolapse. The mesh may be provided with arms for ease of insertion. The arms may be reinforced at the angles of the arms.

The mesh have a knitted borders to maintain the knitted mesh in its format and avoid the fibres being displaced. The knitted borders also help to preserve the shape of the mesh during insertion in the operation and distribute the pulling on the mesh fibres.

The medical device described above can be used in any surgical procedure for repairing vaginal prolapse, such as anterior or posterior vaginal wall repair and vaginal vault prolapse repair, uterine prolapse and also can be used in hernias at other sites of the body.

1. A surgical mesh for use in a surgical procedure for vaginal prolapse repair or repair of hernia, the mesh including a first fiber or yarn and a second Fiber or yarn, wherein the surgical mesh is knitted so that the first fiber or yarn is able to slide over the second fiber or yarn.

2. The surgical mesh of claim 1, the surgical mesh having a porosity at rest of 70% or more.

3. The surgical mesh of claim 1, the surgical mesh having a porosity at rest strictly greater than 75%.

4. The surgical mesh of claim 3, the surgical mesh having a porosity at rest strictly greater than 90%.

5. The surgical mesh of claim 1, wherein the average diameter of a pore at rest is 1 centimeter or more.

6. The surgical mesh of claim 5, wherein the average diameter of a pore at rest is 1.1 centimeter or more.

7. The surgical mesh of claim 1, wherein the surgical mesh is knitted so that the second fiber or yarn is able to slide over the first fiber or yarn.

8. The surgical mesh of claim 1, wherein the first fiber or yarn is joined to the second fiber or yarn by a fixing arrangement or knot.

9. The surgical mesh of claim 1, wherein a pore has a diamond shape, a square shape, a circular shape, any polygonal shape or any irregular shape acquired by the knitting of the fibers.

10. The surgical mesh of claim 1, further including arms for ease of insertion of the surgical mesh during the surgical procedure.

11. The surgical mesh of claim 1, wherein the surgical mesh has a knitted border.

12. (canceled)

13. A process for knitting a surgical mesh for use in a surgical procedure for vaginal prolapse repair or repair of hernia, the process including interweaving a first fiber or yarn with a second fiber or yarn so that the first fiber or yarn is able to slide over the second fiber or yarn.

14. The process of claim 13, wherein the interweaving is such that the second fiber or yarn is able to slide over the first fiber or yarn.

15. A surgical mesh for use in a surgical procedure for vaginal prolapse repair or repair of hernia, the mesh including a first fiber or yarn and a second fiber or yarn, wherein the surgical mesh is knitted so that the first fiber or yarn is joined to the second fiber or yarn by a fixing arrangement or a knot.

16. The surgical mesh of claim 15, wherein the fixing arrangement or a knot constrains a relative movement between the first fiber or yarn and the second fiber or yarn.

17. The surgical mesh of claim 15, the surgical mesh having a porosity at rest of 70% or more.

18. The surgical mesh of claim 15, the surgical mesh having a porosity at rest strictly greater than 75%.

19. The surgical mesh of claim 15, wherein the average diameter of a pore at rest is 1 centimeter or more.

20. The surgical mesh of claim 15, wherein the average diameter of a pore at rest is 1.1 centimeter or more.

21. The surgical mesh of claim 15, wherein the surgical mesh has a knitted border.

22. A process for knitting a surgical mesh for use in a surgical procedure for vaginal prolapse repair or repair of hernia, the process including interweaving a first monfilament or multifilament, absorbable or non absorbable fiber or yarn with a second monfilament or multifilament, absorbable or non absorbable fiber or yarn so that the first fiber or yarn is joined to the second fiber or yarn by a fixing arrangement or knot.

23. The process of claim 22, wherein the fixing arrangement constrains a relative movement between the first fiber or yarn and the second fiber or yarn.

24. A surgical mesh for use in a surgical procedure for vaginal prolapse repair or repair of hernia, the surgical mesh having a porosity at rest of 70% or more.

25. The surgical mesh of claim 24, wherein the porosity is strictly greater than 75%.

26. The surgical mesh of claim 24, wherein the average diameter of a pore at rest is 1 centimeter or more.

27. The surgical mesh of claim 24, wherein the surgical mesh has a knitted border.

28. The surgical mesh of claim 22, wherein the surgical mesh has a knitted border.
